(19) United States
(12) Patent Application Publication
(10) Pub. No.: US 2017/0311810 A1
(43) Pub. Date: Nov. 2, 2017

(54) OBJECT INFORMATION ACQUIRING APPARATUS

(71) Applicant: CANON KABUSHIKI KAISHA, Tokyo (JP)

(72) Inventor: Yoshiko Nakamura, Kusatsu-shi (JP)

(21) Appl. No.: 15/521,936

(22) PCT Filed: Nov. 2, 2015
(86) PCT No.: PCT/JP2015/081421

§ 371 Date: Apr. 26, 2017

(30) Foreign Application Priority Data
Nov. 7, 2014 (JP) 2014-226950

(51) Int. Cl. A61B 5/00

(52) U.S. Cl. CPC A61B 5/0095; A61B 5/7485; A61B 5/4312; A61B 2576/00; A61B 2562/04

(57) ABSTRACT

An object information acquiring apparatus is used, which has: a region setter setting a first region and a second region inside an object; a plurality of probes each receiving an acoustic wave propagated from the object to which light is radiated and outputting an electrical signal; a sound speed determiner setting a plurality of sound speeds for the first region and the second region respectively; an imaging processor acquiring an image using an electrical signal for each sound speed of the first region and acquiring an image using an electrical signal for each sound speed of the second region; a characteristic amount acquirer acquiring a comparison characteristic amount in a common region of the first region and the second region for each sound speed.

### TECHNICAL FIELD

The present invention relates to an object information acquiring apparatus.

### BACKGROUND ART

As an apparatus to image the inside of an object of measurement using an acoustic wave, an apparatus that uses photoacoustic tomography (PAT) has been proposed. A photoacoustic tomography apparatus irradiates an object with laser pulsed light, and using a probe receives a photoacoustic wave which is generated when tissue inside the object absorbs the energy of the irradiated light. Then information related to the optical property values inside the object is imaged using the electrical signal converted from the photoacoustic wave.

When the image is generated, the photoacoustic tomography apparatus uses: the time from the generation of an acoustic wave inside the object into which the light is radiated to the acoustic wave reaching the probe; and the propagation speed (sound speed) of the acoustic wave inside the object. In this case, the information processing is simplified if it is assumed that the sound velocity inside the object is uniform. However if this assumption is considered, contrast and resolution of the image may drop since the sound velocity inside an actual living body is not uniform.

Patent Literature 1 discloses a technique to determine the sound speed in an object based on the dispersion of photoacoustic signals from a specified region, and create an image based on this sound speed.

### CITATION LIST

### Patent Literature

PTL1: Japanese Patent Application Laid-open No. 2011-120765

### SUMMARY OF INVENTION

### Technical Problem

According to Patent Literature 1, the sound speed in a specified region is determined by determining the sound velocity at which phases of the photoacoustic signals before imaging are aligned, and an image is generated based on this sound speed. Therefore when the inside of an object is actually imaged, a subtle difference may be generated in each region, and in some cases partial discontinuity and distortion may be generated.

With the foregoing in view, it is an object of the present invention to generate a good image in photoacoustic tomography by accurately determining the sound speed inside the object.

### Solution to Problem

The present invention provides an object information acquiring apparatus, comprising:

a region setter configured to set a plurality of sub-regions including at least a first region and a second region, which has a common region with the first region, in a region including a region of interest inside an object;

a light source;

a plurality of probes configured to receive an acoustic wave propagated from the object, to which light is radiated from the light source, and output an electrical signal, and receive at least each of an acoustic wave originating from the first region and an acoustic wave originating from the second region;

a sound speed determiner configured to set a plurality of sound speeds for the first region and for the second region respectively;

an imaging processor configured to acquire an image of the first region using the electrical signal with respect to each of the plurality of sound speeds set for the first region, and acquire an image of the second region using the electrical signal with respect to each of the plurality of sound speeds set for the second region;

a characteristic amount acquirer configured to acquire a comparison characteristic amount, which is a characteristic amount in the common region of the image of the first region and the image of the second region, for each of the plurality of sound speeds; and

an information acquirer, wherein

the sound speed determiner acquires first sound speeds of the first region and the second region respectively using the comparison characteristic amount in the common region, and the information acquirer acquires specific information on the object based on the electrical signal and the first sound speeds in the first region and the second region.

### Advantageous Effects of Invention

According to the present invention, a good image can be generated in photoacoustic tomography by accurately determining the sound speed inside the object.

Further features of the present invention will become apparent from the following description of exemplary embodiments with reference to the attached drawings.

### BRIEF DESCRIPTION OF DRAWINGS

FIG. 1 is a diagram depicting a configuration of a photoacoustic tomography apparatus.

FIG. 2 is a schematic diagram of regions which are set by a region setter.

FIG. 3 is a flow chart depicting a first sound speed calculation flow according to an embodiment.

FIG. 4 is a flow chart depicting a second sound speed calculation flow according to an embodiment.

FIG. 5 is a flow chart depicting a processing flow according to Example 1.

FIG. 6 is a diagram depicting a first sound speed distribution according to Example 1.

FIG. 7 is a diagram depicting a second sound speed distribution according to Example 1.

FIGS. 8A to 8C are diagrams depicting an effect of the photoacoustic tomography apparatus according to Example 1.

FIG. 9 is a flow chart depicting a processing flow according to Example 2.

FIG. 10 is a diagram depicting a second sound speed distribution according to Example 2.

### DESCRIPTION OF EMBODIMENTS

Preferred embodiments of the present invention will now be described with reference to the drawings. Dimensions, materials, shapes and relative positions and the like of the components described below should be changed according to the configuration and various conditions of the apparatus to which the invention is applied, and are not intended to limit the scope of the invention to the following description.

The present invention relates to a technique to detect an acoustic wave propagating from an object, and generate and acquire specific information on the interior of the object. Therefore the present invention is understood as an object information acquiring apparatus, a control method thereof, an object information acquiring method, and a signal processing method. The present invention is also understood as a program that causes an information processing apparatus, which includes such hardware resources as a CPU, to execute these methods, or a storage medium storing this program. The present invention is also understood as an acoustic wave measurement apparatus and a control method thereof.

The object information acquiring apparatus of the present invention includes an apparatus utilizing a photoacoustic tomography technique that irradiates an object with light (electromagnetic wave) and receives (detects) an acoustic wave, which is generated inside or on the surface of the object according to the photoacoustic effect, and is propagated. The object information acquiring apparatus, which acquires the specific information on the interior of the object in the format of, for example, image data based on the photoacoustic measurement, can also be called a “photoacoustic imaging apparatus” or a “photoacoustic tomography apparatus”.

The specific information in the photoacoustic apparatus is a generation source distribution of an acoustic wave generated by radiating the light, an initial sound pressure distribution inside the object, a light energy absorption density distribution and an absorption coefficient distribution derived from the initial sound pressure distribution, or a concentration distribution of a substance constituting a tissue. In concrete terms, the concentration distribution is: an oxy/deoxyhemoglobin concentration distribution, a blood component distribution such as the oxygen saturation distribution determined from the oxy/deoxyhemoglobin concentration; or a distribution of fat, collagen, water and the like. The specific information may be determined not as numeric data, but as a distribution information at each position inside the object. In other words, the object information may be the distribution information, such as the absorption coefficient distribution and the oxygen saturation distribution.

An acoustic wave referred to in the present invention is typically an ultrasound wave, and includes an elastic wave which is also called a “sound wave” or an “acoustic wave”. An acoustic wave generated by the photoacoustic effect is called a “photoacoustic wave” or a “light-induced ultrasound wave”. An electrical signal converted from an acoustic wave by a probe is called an “acoustic signal”, and an acoustic signal originating from a photoacoustic wave in particular is called a “photoacoustic signal”.

An object in the present invention can be a breast of a living body. The object, however, is not limited to this, but may be other segments of a living body, or a non-biological material.

(Basic Example)

Using a basic example of the present invention, the configuration and functions of the apparatus and the processing flow will be described.

<Apparatus Configuration>

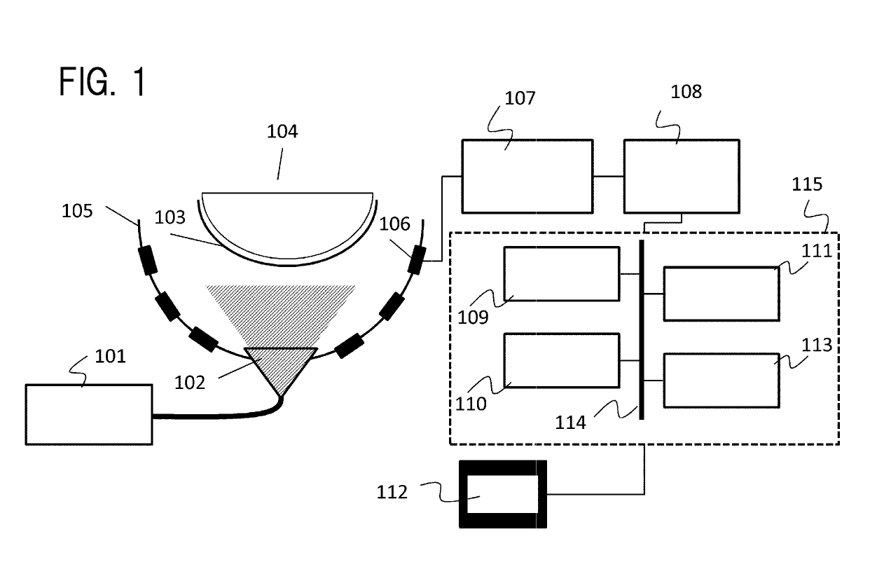

In FIG. 1, a photoacoustic tomography apparatus has: a light source 101, a light irradiation unit 102, an acoustic matching material 103, a probe array 105 and probes 106, and a probe scan drive unit and a scan controller which are not illustrated. An object 104 is a measurement target. The photoacoustic tomography apparatus also has: a signal processor 107, a signal memory 108, an imaging processor 109, a region setter 110, a sound speed determiner 111, a display unit 112, and a characteristic amount acquirer 113. It is preferable to include an input unit for an operator to input instruction content and to specify numeric values.

(Light Irradiation)

The light source 101 generates pulsed light, and irradiates the object 104 with the pulsed light using the light irradiation unit 102. A photoacoustic wave is generated from an absorber inside or on the surface of the object to which the pulsed light is radiated.

It is preferable that the light source 101 can generate pulsed light in a nano second order. The wavelength of the irradiation light is preferably 700 nm or more, with which the light cannot be absorbed by hemoglobin, collagen or the like, so that the light can reach a deep region inside the object. For the light source 101, a wavelength-variable laser, which can change wavelength according to the constituent to be imaged, is preferable since the light absorption spectrum is different depending on the constituent inside the object.

For the light source 101, laser is desirable because of its high output, but a light emitting diode or the like may be used instead of laser. For the laser, various lasers can be used, such as a solid-state laser, a gas laser, a dye laser and a semiconductor laser. The timing of irradiation, waveform, intensity or the like is controlled by a light source controller, which is not illustrated.

Optical members to guide the light from the light source to the object may be included, such as: a mirror to reflect light, a lens to collect or expand light or to change the shape of the light; a prism to disperse, refract or reflect light; an optical fiber to propagate the light; and a diffusion plate. The light irradiation unit 102 is disposed on the edge of these optical members.

(Acoustic Wave Reception)

The probe 106 on the probe array 105 receives a photoacoustic wave propagated through the acoustic matching material 103. The probe 106 is a detector which includes one or more elements to receive the acoustic wave. If a plurality of elements are arranged on the surface of the probe, a signal can be simultaneously acquired at a plurality of positions. Thereby the reception time can be decreased, the influence of vibration of the object can be reduced, and the SN ratio can be improved.

The probe 106 receives an acoustic wave, converts it into an electrical signal, and outputs the electrical signal. An element used for the probe can be a conversion element using the piezoelectric phenomenon, a conversion element using the resonance of light, a conversion element using the change of capacitance, or the like. However the present invention is not limited to these elements, only if the element can receive an acoustic wave and convert it into an electrical signal.

The probe array 105 has a hemispheric structure. A support member having a certain strength is preferable, since a plurality of probes 106 are arranged on the probe array. It is preferable to dispose the probes 106 spirally. In the example in FIG. 1, a light irradiation unit 102 is disposed on the pole at the bottom of the hemisphere. It is preferable that a solution for acoustic matching, such as water and caster oil, fills the inside of the hemisphere.

It is preferable that the probe array 105 can rotate around the rotation axis passing through the light irradiation unit 102. In this case, the light source 101 radiates a plurality of light pulses in sync with the rotation of the probe array 105. The plurality of probes 106 receives each photoacoustic wave, which is generated by each light pulse, at a plurality of positions around the object. This configuration is preferable since acoustic waves can be received from the entire area around the object using a limited number of probes 106.

By disposing a plurality of probes 106 on the hemispheric surface, a region, where the high sensitivity direction (reception directivity) of each probe 106 concentrates in an area near the center of the hemisphere, is generated. This region is called a “high resolution region”, where inside of the object can be imaged at high precision.

The shape of the probe array 105 is not limited to a hemisphere. For example, the shape of the probe array 105 may have a spherical crown shape, a part of an ellipsoidal or a combination of a plurality of planes or curved surfaces. It is required, however, that the shape is appropriate to support the plurality of probes so that the directivity axes of at least part of the plurality of probes concentrate.

The rotation position of the probe 106 is controlled by the scan drive unit, so as to be in sync with the emission of the light source 101. It is preferable that the probe array 105 can move with respect to the object. The motion of the probe array 105 itself caused by the scan drive unit is preferably translational (in a horizontal direction and depth direction in the drawing of FIG. 1). By this scanning, the high resolution region is moved within the object, and the entire object can be imaged at high resolution. The timings of the scan of the probes and signal acquisition are controlled by the scan controller.

A holding member to support the object may be disposed on an upper part of the probe array. This allows, for example, the shape of the object to be maintained and estimation of the light quantity distribution inside the object to become easier. The preferable shape of the holding member is a cup shape or a bowl shape. It is preferable that the holding member is transmissive to the irradiated light and the photoacoustic wave from the inside of the object.

(Signal Processing)

The signal processor 107 performs digital conversion and amplification processing on an electrical signal originating from the acoustic wave. The signal processor averages the signals acquired at the same position in the same direction, and outputs the result. It is preferable that the signal processor uses a bandpass filter for the received signals so as to reduce noise. Any circuit or information processing apparatus having this function can be used as the signal processor.

The signal memory 108 records the signal outputted from the signal processor. The signal memory 108 records an electrical signal which the probe 106 outputs at each emission of the light source 101 according to the scanning in the rotational and translational directions of the probe array 105. For the signal memory, a conventional circuit or a component having the required functions, such as an FIFO memory, can be used.

(Region Setting)

The region setter 110 sets a region of interest and sub-regions. The region of interest is a region in the object, in which specific information is determined, and is determined according to the design values based on the configuration of the apparatus and the range specified by the user. Normally the region of interest is a target of imaging, and therefore may be called a “display image region”. The sub-region is a region in which the optimum sound speed of the image is calculated. In a region that includes the region of interest, the region setter 110 preferably sets regions so that each sub-region has a common region, where a sub-region overlaps with at least another sub-region.

The size of the sub-region is set according to: the sound speed distribution, the apparatus resolution, the sound speed setting range of the sound speed determiner, the distance between the probe and the absorber (probe-absorber distance) and the like. The shape of the sub-region is not limited to a square, and can be any shape, such as a circle.

For comparing image characteristic amounts, it is preferable that the image quality within a sub-region is uniform. Hence it is better if the region setter sets a sub-region in a range where the sound speed is approximately uniform. For example, a tissue where the sound speed is the same continues for about 50 mm in a certain portion in the object, then one side of the sub-region is set to 50 mm or less.

Further, because the sound speed is calculated based on the image in the sub-region according to the present invention, the size of the sub-region is set to be sufficiently wider than the resolution of the apparatus. If one side of the sub-region is set to a distance that can be recognized by the resolution of the apparatus, the accuracy of acquiring the image characteristic amount drops since it is difficult to discern whether the signal in the image is a noise, a blur, or whether it comes from an absorber.

In the following description, two sub-regions (corresponding to the first region and the second region of the present invention) are selected out of a plurality of sub-regions which are set in a region that includes the region of interest, and are compared. However, selecting and comparing three or more sub-regions at the same time is also included in the scope of the present invention.

Even if a same absorber is imaged, the position of the absorber in the image changes depending on the sound speed that is set by the sound speed determiner. This positional change becomes conspicuous when the sound speed between the probe and the absorber is uniform. For example, it is assumed that the absorber is located 130 mm from the probe, and the sound speed between the absorber and the probe is 1450 m/s. In this case, if the sound speed determiner sets the sound speed to 1400 m/s, the position of the absorber on the image becomes 125.5 mm, while if the sound speed determiner sets the sound speed to 1500 m/s, the position of the absorber becomes 134.5 mm. In such a case, to cover the positional shift generated by the change of the sound speed setting, it is preferable to set the size of the sub-region to 10 mm or more.

The region setter also selects an electrical signal for each sub-region to be used for creating an image of the sub-region. In this case, it is preferable to select an electrical signal which is very likely originating from a photoacoustic wave emitted from the absorber in the sub-region, according to the reception sensitivity, the directional angle or the like of the probe. Generally the image characteristic amount can be accurately acquired when the change of the sound speed on the propagation path of the photoacoustic wave, from the sound source to the probe in the sub-region, is small. Therefore it is preferable that the acoustic wave acquisition positions used for the image reconstruction of one sub-region are not dispersed very much in the sub-region.

If an electrical signal used for imaging a sub-region is used for imaging another sub-region, the images in the common region of these sub-regions become the same as the images of the sub-regions. As a result, the image characteristic amount used to compare with the common region becomes a maximum, which diminishes the significance of the comparison. Therefore it is preferable that the same signals are not used for imaging two sub-regions that share a common region, this means that electrical signals acquired under different conditions are used for the two sub-regions.

However, even if the acoustic waves used for reconstructing images of both sub-regions originate from the same light irradiation, it is still meaningful to compare the images because of, for example, the difference of the transfer path of the acoustic wave inside the object. Therefore if a same signal is used for imaging sub-regions that include the common region, it is preferable to weigh the image characteristic amount of each sub-region image.

When a sub-region and an adjacent sub-region thereof are imaged and compared, it is also effective to change the pattern of the probes to be used. In this case, it is preferable to select and create a pattern of probes that can image a sub-region at high sensitivity based on the distance and angle of the sub-region. The later mentioned region setter may determine the probe pattern in advance for each sub-region when the object is divided into sub-regions.

Furthermore, the position of the high resolution region of each probe inside the object moves according to the scanning by the scan drive unit. Therefore, for each sub-region, the region setter may set a segment inside the object that corresponds to the high resolution region at each scanning position. In this case, an advantage is that the reconstructed image used for comparing adjacent sub-regions can be acquired at high sensitivity.

For the acoustic waves used for reconstructing images of both sub-regions, acoustic waves acquired by different light irradiation may be used.

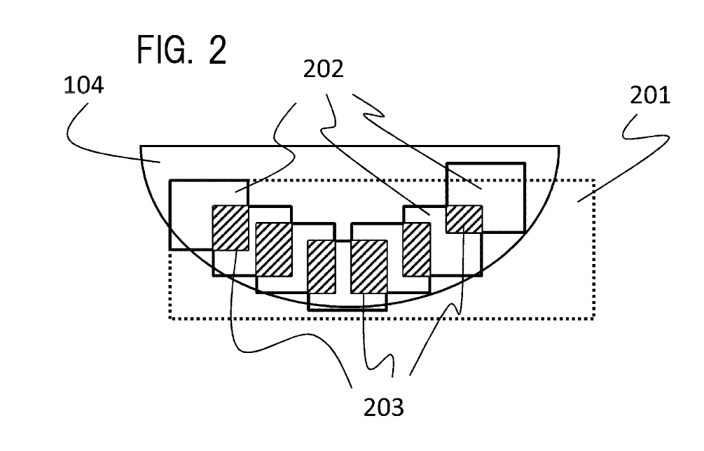

FIG. 2 shows the regions which are set by the region setter 110. The region setter determines the region of interest 201 that includes the object region. Then the region setter divides the region that includes the region of interest 201 into a plurality of sub-regions 202. The sub-regions 202 are set so as to have a common region 203 with at least one adjacent sub-region. Each region is imaged by the imaging processor. The imaging sound speed required for imaging is preferably outputted from the sound speed determiner. As illustrated here, a sub-region may extend from the region of interest. Also sub-regions may not be disposed some areas in the region of interest. In this case, the later mentioned interpolation processing is effective.

(Imaging)

The imaging processor 109 images the sub-regions using the sound speed that is set and the electrical signals selected by the region setter out of the electrical signals stored in the signal memory 108. The imaging method can be, for example, a universal back projection, a back projection in a Fourier domain, an aperture synthesis method or a time reversal method. Other image reconstruction logics may be used. Thereby the specific information (e.g. absorption coefficient distribution) in the sub-regions can be acquired. Here “imaging” refers to creating image data to be the source of the display image. This image data reflects the specific information on the object, hence the imaging processor according to the present invention may be called an “information acquirer” that creates volume data originating from the specific information. The image processor not only creates an image used for extracting the characteristic amount in the intermediate processing steps, but also creates a final display image for the user. Therefore the imaging processor plays a role of the information acquirer of the present invention as well.

For the imaging, a plurality of sound speeds may be set for one sub-region. For example, if water is filled outside the object, two sound speeds, (the sound speed of water outside the object and the sound speed inside the object) may be specified. Then the propagation path is divided, and an appropriate sound speed is used for each divided section of the path. If there is an object in which the sound speed is different from both the object and the matching material, such as a holding plate that holds the shape of the object, the setting for this object may be added.

(Characteristic Amount Acquisition and Sound Speed Determination)

Sub-region images created by the imaging processor are inputted to the characteristic amount acquirer. Then using the image characteristic amounts outputted from the characteristic amount acquirer, the sound speed determiner compares the image characteristic amounts of the sub-regions and the common regions where sub-regions overlap, and determines the optimum sound speed. This processing includes a first sound speed calculation processing to determine a first sound speed with which the image characteristic amount of each sub-region becomes the optimum, and a second sound speed calculation processing to determine a second sound speed with which the image characteristic amount of the common region becomes the optimum.

(First Sound Speed Calculation Processing)

The sound speed determiner 111 calculates the image sound speed of each sub-region 202 first. Then the characteristic amount acquirer calculates the image characteristic amount based on the created sub-region image. The image characteristic amount is an index or a combination of indexes that represents the characteristics of the sub-region image. For example, the image characteristic amount represents the resolution of the image, contrast, edge sharpness, image intensity, shift amount between images or the like. The contrast is, for example, a ratio of the maximum value and the minimum value of the image intensity in the imaged region, or a ratio of the averages of image intensities in the absorber and the background range specified by the operator.

The sound speed determiner calculates a first sound speed with which the image characteristic amount becomes a predetermined value, and records the first sound speed. The first sound speed is an optimum sound speed in a single sub-region image.

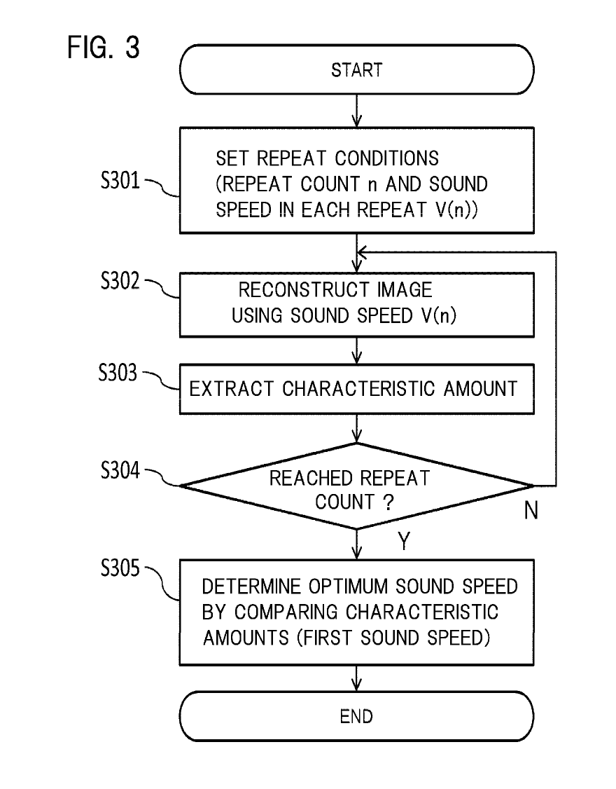

The first sound speed calculation processing will be described in concrete terms with reference to the flow chart in FIG. 3. The processing of this flow starts when the photoacoustic wave propagated from the object is received by the probe array and is saved in the memory. It is preferable that the photoacoustic wave is received a plurality of times.

After the start of processing, repeat conditions are set for a sub-region in step S301. Here a repeat count n and sound speed V(n), which is used for image reconstruction in each repeat, are set.

In step S302, the imaging processor 109 images the sub-region using the electrical signal stored in the signal memory 108 and the sound speed which is set, and inputs the image into the sound speed determiner.

Then in strep S303, the characteristic amount acquirer calculates the image characteristic amount from the inputted image. This processing may be performed in advance before comparison processing. If it is determined that the repeat count is reached in step S304, then processing advances to step S305.

In step S305, the sound speed determiner compares the characteristic amounts of the images acquired using each sound speed, determines the first sound speed in the pressing target sub-region, and then records this result.

The method for determining the first sound speed is not limited to the method of the flow in FIG. 3. For example, if the first sound speed can be estimated by such a method as image analysis, the image characteristic amount may be calculated based on this first sound speed.

In FIG. 3, the sound speeds are determined in advance, and the image characteristic amount is determined for all the sound speeds. However the flow in FIG. 3 may not always be adhered to. In other words, it is also possible that the characteristic amount is repeatedly extracted while the sound speed is gradually changed from the initial value, and the sound speed when the characteristic amount reached a predetermined threshold is used.

In this case, the amount of change of the sound speed is estimated using the amount of change of the image characteristic amount from the previous sound speed. So in this case, a bisection method, a Newton's method, a steepest descent method, a minimum gradient method, a golden section method, a direct search method or the like, which are used as the optimization processing method, can be used.

The sound speed determiner typically compares the total of the image characteristic amounts in all sub-regions or common regions. However, the sound speed determiner may compare the average value of the image characteristic amounts, or compare the image characteristic amounts in specific pixels of a region or in a peripheral range around a specific pixel. “Specific pixel” here refers to a pixel having an intensity that seems to be originating from an absorber in a sub-region or a pixel of which intensity is highest in a sub-region.

In the photoacoustic tomography, oxygen saturation distribution, glucose distribution, collagen distribution or the like can be acquired by comparing absorption coefficient images having different wavelengths. Specific pixels may be determined based on this specific information on the object.

If there are few absorbers in a sub-region, then noise and artifacts in the images occupy most of the area of the image. In such a case, the image characteristic amount may be affected by the noise and artifacts. If it is determined that a predetermined amount or more of an absorber does not exist in a sub-region, it is preferable not to calculate the optimum sound speed in the sub-region, or to compare the image characteristic amounts in the peripheral area of the pixels of the absorber.

(Second Sound Speed Calculation Processing)

By applying the flow in FIG. 3 to each sub-region, the first sound speeds are determined for all the sub-regions. Then the image is reconstructed for each sub-region using the respective first sound speed, and these reconstructed images are simply combined, whereby an image in which the sound sped is optimized for each segment of the object is acquired, and the effect of the present invention is demonstrated. In this sense, the first sound speed corresponds to the optimum sound speed according to the present invention.

However, if a simple combination processing is performed, the cross-sections of the absorber become discontinuous in the boundaries of the image, and strains are generated in the image. Further, when the common regions are created, the absorber image may be seen as double or blurred, depending on the image processing method. Therefore, in order to align the absorber images in the common portions, it is preferable to search sound speeds in a plurality of sub-regions, including the common portions, and determine an optimum sound speed (second sound speed) of imaging in each sub-region.

Now a second sound speed calculation processing, to prevent a sudden change of the image in the boundary portions and a drop of image quality in the common regions, will be described. The second sound speed is the sound speed with which the image characteristic amount in a common region, extending over a plurality of sub-regions 202, satisfies the optimum conditions. The second sound speed is also determined for each sub-region, just like the first sound speed, and is used for reconstructing the image of the sub-region. A method that can be used for creating an image of a common region is, for example, a method of reconstructing an image using an electrical signal and sound speed of one of the overlapping two regions, a method of reconstructing an image after determining an intermediate sound speed between the sound speeds of two regions, a method of combining two images or the like.

The imaging processor images each sub-region while changing the sound speed by a predetermined value at a time, based on the first sound speed in each sub-region as a reference. The characteristic amount acquirer acquires the characteristic amount of each image, and the sound speed determiner compares the characteristic amounts of the images acquired using a plurality of sound speeds among adjacent sub-regions. Thereby a second sound speed, with which the image characteristic amount becomes the optimum, can be acquired for each sub-region.

The image characteristic amount to determine the second sound speed is, for example, a value that indicates the shift of each sub-region image in the common region. This value may be called the “second characteristic amount” since this value is compared with the characteristic amount to determine the optimum sound speed in each sub-region (first region and second region). If the shift of images in the common region decreases, the strain between the sub-regions in the display image also decreases. The shift of each sub-region image is determined by extracting and comparing the characteristic amount of the common region image of each sub-region image, for which a matching method, based on, for example, normalized cross-correlation, can be used. The optimum conditions are conditions to improve the image quality of the entire display image.

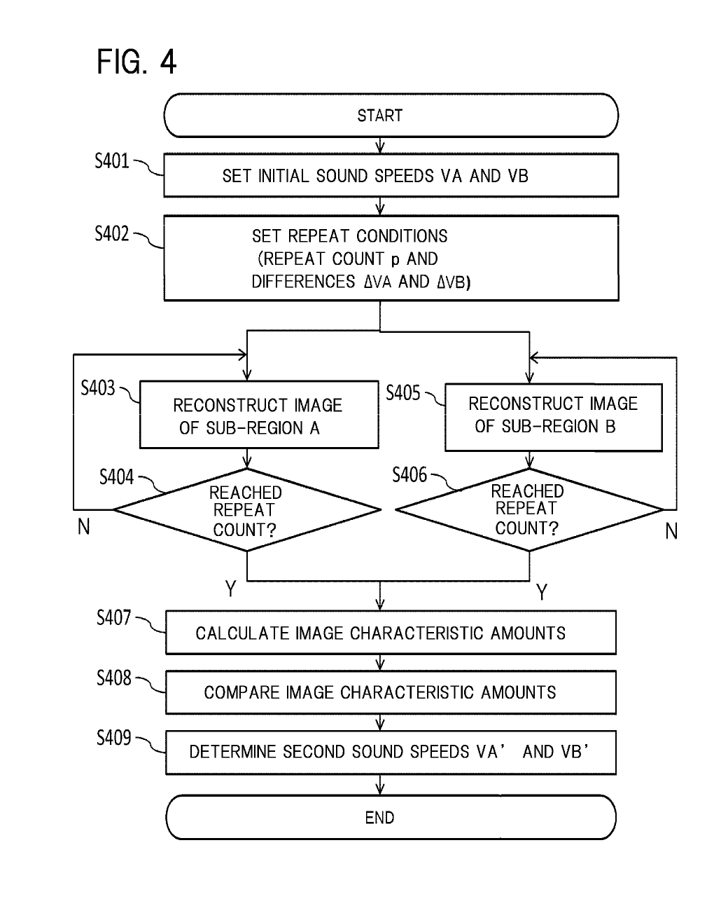

The processing procedure to determine the second sound speed will be described with reference to the flow chart in FIG. 4. It is assumed that the first sound speed in each sub-region has already been determined when this flow starts, and is VA in sub-region A and VB in sub-region B. These first sound speeds are set in the apparatus in step S401. It is assumed that the second sound speeds in the sub-region A and sub-region B, to optimize the common region image, are VA′ and VB′ respectively, which are determined by this flow.

In step S402, the processing repeat condition in each sub-region is set. The repeat count is assumed to be p, and an increment (difference) of the sound speed in each repeat time is assumed to be ΔVA and ΔVB. In this basic flow, the repeat count is set as a condition, but the repeat processing may be terminated when the image characteristic amount of the common region image satisfies a predetermined optimum condition. Instead of predetermining ΔVA and ΔVB, the image characteristic amount may be determined in each repeat time based on the increase/decrease amount of the image characteristic amount in a previous time. At each repeat, VA =VA+ΔVA and VB=VB+ΔVB are inputted to the imaging processor as the sound speed values.

If the difference of the first sound speed in the sub-region A and in the sub-region B is large, the second sound speed in each region is more likely to be an intermediate value between these first sound speed values. Therefore, if the sound speed of the sub-region A is higher when ΔVA and ΔVB are determined, for example, it is possible to set the value of ΔVA in the descending direction and the value of ΔVB in the ascending direction.

The imaging processor images the sub-region A using the current VA, and the sub-region B using the current VB respectively (steps S404, S405). The created images are inputted to the sound speed determiner. If the imaging processing reaches a predetermined number of times (steps S404, S406), processing advances to the next processing. In other words, in this flow, an image generated using a sound speed that is slightly changed at each repeat time based on the first sound speed as a reference, is generated and saved for each sub-region.

The characteristic amount acquirer calculates the image characteristic amount at least for the common region using the reconstructed image with each sound speed. The sound speed determiner compares the image characteristic amounts in the common region (steps S407 to S408). The image characteristic amount is, for example, and index value that indicates the difference of pixel intensities of the images, the shift amount between images, or the degree of correlation between the images, in the common region where the sub-region A and the sub-region B overlap. This characteristic amount can be called the “comparison characteristic amount” among the plurality of sub-regions (e.g. first region and second region). The comparison characteristic amount can be acquired by determining and comparing the individual characteristic amount of each sub-region. The individual characteristic amount is, for example, resolution, contrast, edge sharpness, image intensity or the like.

Hereafter the common region image on the sub-region A side is denoted with I_{A}, and the common region image on the sub-region B side is denoted with I_{B}. The image characteristic amount in a common region includes the characteristic amount calculated by comparing the common region portions of the sub-regions. The image characteristic amount in the common region is, for example, a difference of the image characteristic amounts in I_{A} and I_{B}, a shift amount of these images or the like. The image characteristic amounts may be compared using all of the plurality of images acquired repeatedly, or appropriate images may be selected based on a predetermined standard, and comparison may be performed using only these images.

The sound speed determiner determines the maximum amounts of ΔVA and ΔVB. The maximum amounts may be set as concrete sound speed values (e.g. 10 m/s). Thresholds may be set for the image characteristic amounts of sub-regions that are calculated using ΔVA and ΔVB. The sound speed determiner sets a plurality of ΔVA and ΔVB, and reconstructs the sub-regions using the respective sound speed values, and calculates the image characteristic amounts. Then the sound speed determiner determines the second sound speeds ΔVA′ and ΔVB′ at which the characteristic amounts of the common region portions become the optimum (step S409).

(Image Display)

The imaging processor creates a display image by combining the sub-region images generated using the second sound speeds, and displays this image on the display unit 112. In this case, the border lines of the sub-regions and the sound speed (at least one of first sound speed and second sound speed) in each sub-region may be displayed as well. Thereby the discontinuity and strain among the sub-regions 202 can be reduced.

Various methods can be used for the imaging processor to combine the sub-region images in the common region. For example, averaging the pixel values, a method of using the pixel value of an appropriate sub-region for each pixel or the like can be used.

If there is a region of interest that is not included in the sub-region image, the imaging processor estimates the sound speed value based on the second sound speed values of adjacent sub-regions, so as to interpolate the image. Instead the imaging processor may estimate the optimum sound speed in the entire region of interest based on the optimum sound speed in each sub-region, and create an image of the entire region of interest using this optimum sound speed.

In the above mentioned flow, combining two sub-regions was described, but the second sound speed can be determined sequentially even if there are three or more sub-regions. For example, a case when there are sub-region A, sub-region B which is adjacent to sub-region A, and sub-region C which is not adjacent to sub-region A but is adjacent to sub-region B will be described. In this case, the second sound speeds are determined for sub-region A and sub-region B using the method in FIG. 4, and then the second sound speeds are determined for sub-region B and sub-region C. Then two second sound speeds are calculated for sub-region B. In this case, a sound speed in any one of the sub-regions may be fixed so that the sound speeds in the other sub-regions are determined based on [the fixed sound speed]. Alternatively, the second sound speed in sub-region B may be determined such that the second sound speeds of the three sub-regions are balanced.

In this flow, an image is created for the candidate values of the second sound speed respectively. However, a reconstructed image that is created to determine the first sound speed in each sub-region may be saved in advance, so as to be used for calculating the second sound speed.

Further, in this flow, the optimum sound speed in each small region and the optimum sound speed in the common region are determined independently. However, if the processing to determine the first sound speed from the image characteristic amount in each sub-region and the processing to determine the second sound speed using the image characteristic amount in the common region are combined, the optimum sound speed in the display image can be determined in one processing flow.

The imaging processor, the region setter, the sound speed determiner and the characteristic amount acquirer may be configured by dedicated circuits respectively, or may be implemented using the information processing apparatus that has a processor and operates according to the program, as shown in FIG. 1. Here each block is schematically shown, and these components may be configured as a single unit. Alternatively, these components may be divided into smaller modules according to the respective function. In the example in FIG. 1, the processor (not illustrated) of the information processing apparatus 115 (indicated by the broken line) functions as the controller, and controls each block connected via the bus 114.

The created display image is displayed on the display unit. For the display, an MIP (Maximum Intensity Projection) image or a slice image is preferable. A method of displaying a 3D image from a plurality of different directions may also be used. It is preferable to dispose a user interface, whereby the user can change the inclination of the display image, display region, window level or window width while checking the display.

On the display unit, a display image, an optimum sound speed distribution in the sub-region, an estimated optimum sound speed distribution, and a sub-region image selected by the operator, for example, can be displayed. These images may be displayed simultaneously.

### Example 1

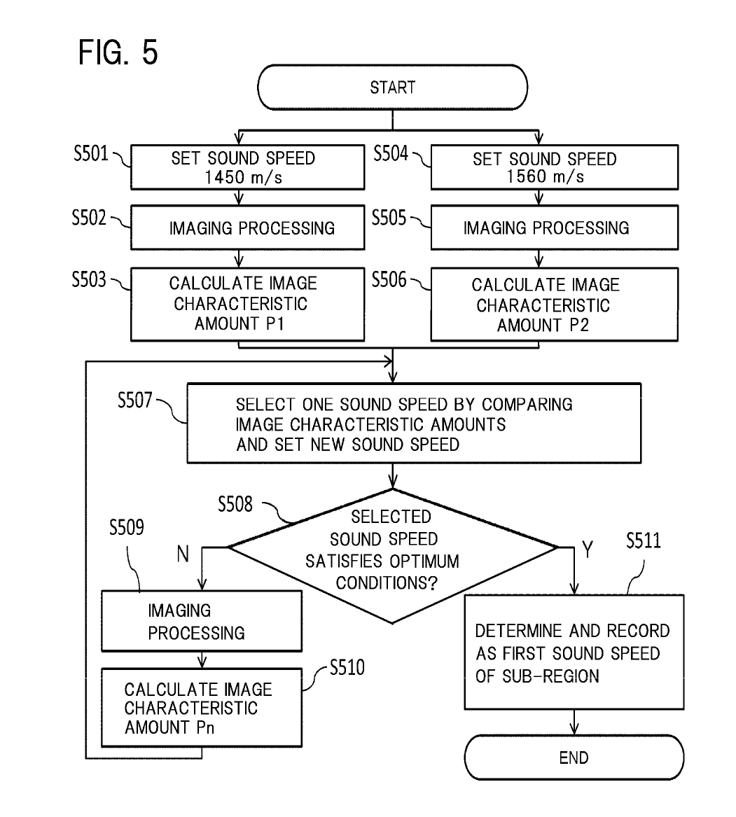

FIG. 5 shows a processing flow of a first sound speed calculation in a sub-region. In this example, the above mentioned method of setting the repeat count and the sound speed in each time in advance is not used. Instead the processing of selecting the optimum sound speed from two sound speeds is continued until a sound speed that satisfies a predetermined condition is acquired.

First the sound speed determiner sets 1450 m/s and 1560 m/s (steps S501, S504). Then the imaging processor creates images of the sub-regions using these sound speeds (steps S502, S505). Here MIP images are created in the x, y and z directions respectively based on the intensity of the specific information acquired by the imaging processor.

Then in steps S503 and S506, the characteristic amount acquirer calculates the first order differential values in two directions of each MIP image, and acquires the image characteristic amounts P1 and P2 by calculating the total of the absolute values of the acquired differential values. This image characteristic amount corresponds to the edge sharpness of the image. As the image sound speed approaches the optimum sound speed, the boundary of the absorber in the image becomes clearer, and the edge sharpness of the image improves. When the characteristic amounts in all three directions are acquired for each sound speed, processing advances to the next processing.

In step S507, the sound speed determiner compares the image characteristic amounts P1 and P2, and selects a sound speed by which a better value is acquired. Then the sound speed determiner sets a new sound speed instead of an unselected sound speed. At this time, a method of changing the sound speed at 2 m/s intervals from the selected sound speed as a reference within a±10 m/s range can be used, for example. The new sound speed value may be set so that the sound value approaches the selected sound speed value from the unselected sound speed value by a predetermined value. If the image characteristic amount acquired from the new sound speed value set like this is lower than the image characteristic amount acquired from the previous sound speed value, the previous sound speed value can be used.

Then it is determined whether the sound speed on the selected side satisfies the predetermined optimum conditions in step S508. Here the optimum sound speed is expressed by the following Expression (1).

$$\begin{matrix}[ Math . 1 ] & \\ \hat{V} = {max}_{v} ( \vert \frac{\partial }{\partial y \partial z} {I}_{vx} \vert + \vert \frac{\partial }{\partial z \partial x} {I}_{vy} \vert + \vert \frac{\partial }{\partial x \partial y} {I}_{vz} \vert ) & ( 1 )\end{matrix}$$

Here Iv denotes an example of a sub-region image that is imaged with sound speed v. Ivx, Ivy and Ivz denote the MIP images in the x, y and z directions respectively. To efficiently find the optimum sound speed in the sub-region, a golden section method, for example, can be preferably used. Here it is assumed that the optimum sound speed is determined at a 1 m/s or less accuracy. In this example, the MIP images in the three directions are used, but a three-dimensional data image may be used directly.

In S508, the sound speed determiner determines whether the selected sound speed is within a predetermined range according to the optimum sound speed condition. The processing to set a new sound speed in S507 may be performed only when this condition is not satisfied. Then in steps S509 and S510, the sub-region is imaged with the new sound speed, and the characteristic amount Pn is extracted. Then the characteristic amount comparison processing is executed again.

As an example of the processing operations in steps S507 and S508, the sound speed determiner may set a new sound speed by using a golden section method, and determine whether the repeat condition is satisfied. An example of the repeat condition is whether or not the absolute value of the difference of the two sound speeds is 1 [m/s] or less.

In this example, this processing is repeated until the sound speed that satisfies the optimum second speed condition is acquired. Then in step S511, the optimum sound speed is determined as the first sound speed, and this sound speed is recorded.

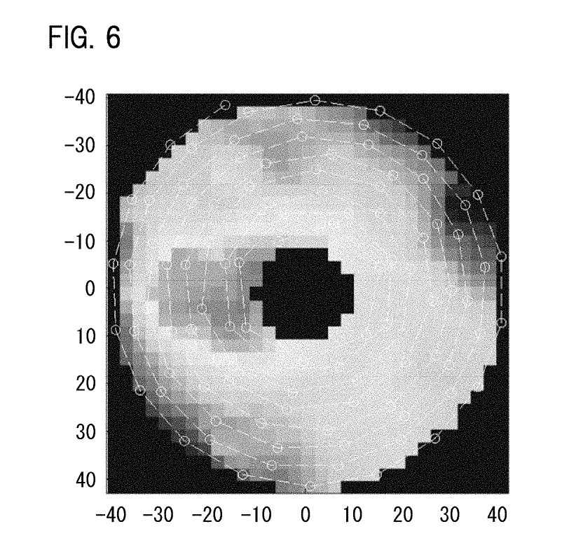

FIG. 6 shows the optimum sound speed distribution (first sound speed) in each sub-region calculated as above. A small white circle in FIG. 6 indicates a center coordinate of each sub-region that is set, and the grayscale indicates the optimum sound speed value.

Then the sound speed determiner calculates the image characteristic amount in the common region between a sub-region and an adjacent sub-region, and compares these image characteristic amounts so as to calculate the second sound speed. In this case, the image characteristic amount in the common region is preferably the shift amount of the absorber that exists in the common region. The shift amount is the amount by which the normalized cross-correlation value increases. The normalized cross-correlation value is a representative example of the index value to determine the shift amount, and another correlation value may be used instead.

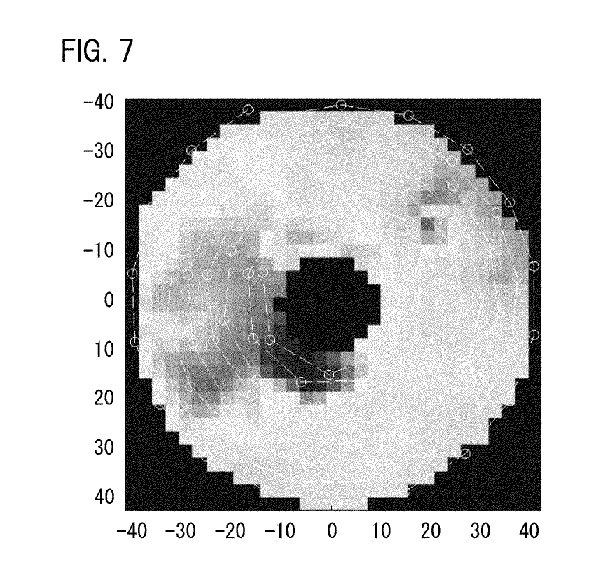

In this example, the second sound speed, which is the optimum sound speed for the display image, is determined sequentially from the sub-region closest to the center. Under a condition where each second sound speed is not changed once determined, the second sound speed is determined for all sub-regions. FIG. 7 shows the second sound speed distribution. However depending on the operation capability of the information processing apparatus, a previously determined second sound speed may be corrected by feedback from a second sound speed determined in another location, so as to balance the entire display image.

The imaging processor reconstructs the image in each sub-region based on the respective second sound speed, and creates the display image by combining each sub-region image. The created image data may be stored in the storage apparatus, or may be transmitted to and displayed on the display unit.

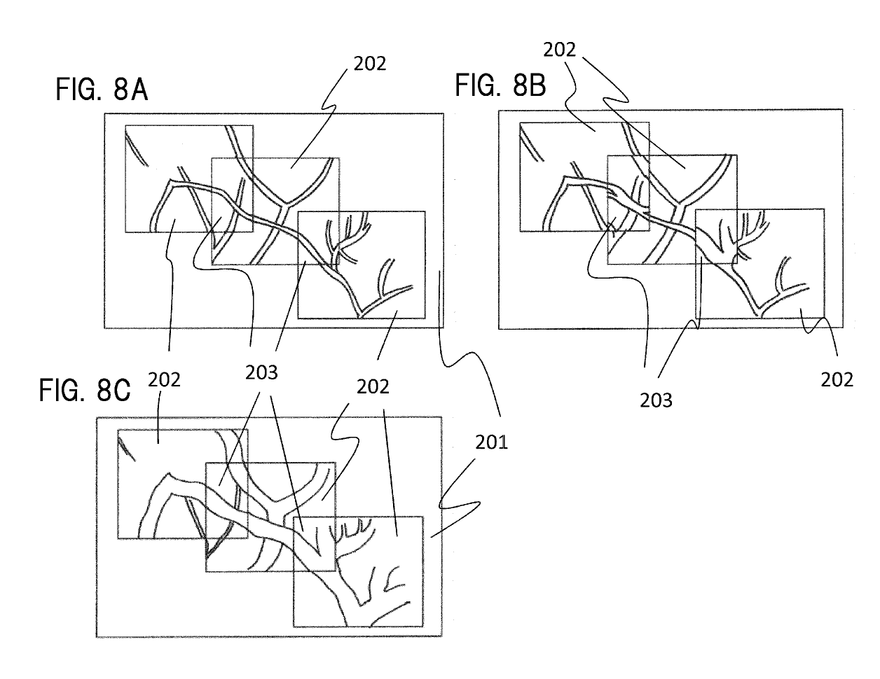

FIG. 8 shows the schematic diagrams depicting combined images. FIG. 8A is an image when the sub-regions imaged with a second sound speed are combined. FIG. 8B is an image when sub-regions imaged with the first sound speed are combined. And FIG. 8C is an image when the sub-regions imaged at 1530 m/s, which is typically used as the sound speed in a living body, are combined.

FIG. 8B, which used the first sound speed, has a better resolution compared with FIG. 8C, which used a general sound speed. In FIG. 8A which used the second sound speed, a shift and blur of the absorbers located between the boundary of the sub-region 202 and the common region decreased, compared with FIG. 8B which used the first sound speed. In this way, it is confirmed that resolution of the absorber in the object in the display image improves if the optimum sound speed is determined for each sub-region and used for imaging. Further, it is confirmed that not only does resolution improve, but also the unnatural display in the boundary portion can be reduced if an image is generated using the second sound speed determined by comparing the sub-regions in the common region.

### Example 2

In the above example, the optimum sound speed in the sub-region image is determined first, then the optimum sound speed in the display image is determined considering the image characteristic amount in the common region. In Example 2, the image characteristic amounts of the sub-region and the common region are calculated simultaneously. Thereby time required for determining the optimum sound speeds can be decreased. An aspect that is different from Example 1 will be described in detail.

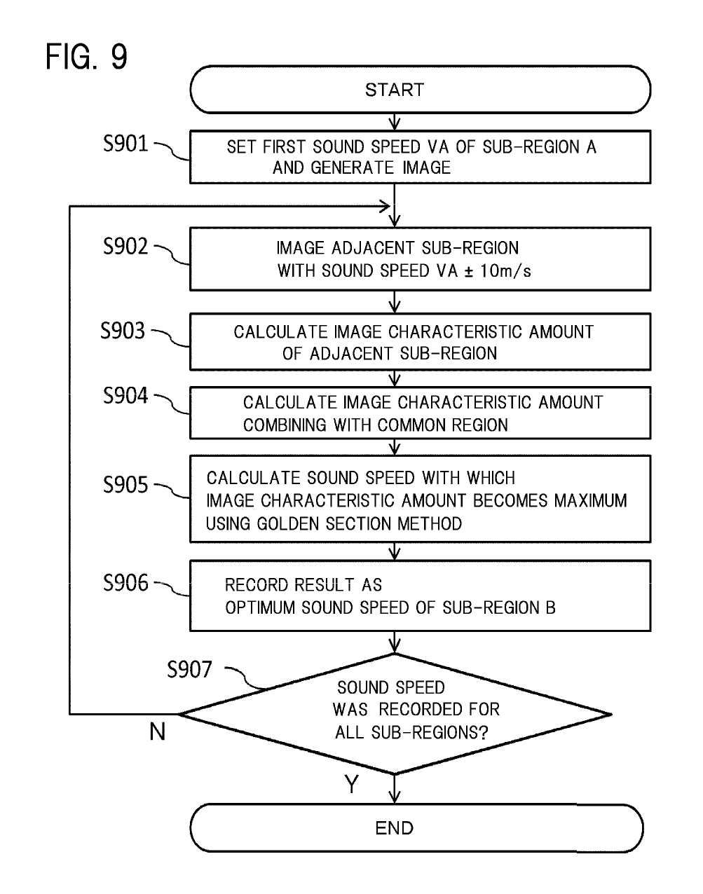

FIG. 9 shows the processing flow of this example. First in step S901, the sound speed determiner determines the optimum first sound speed VA in sub-region A. Here it is assumed that 1510 m/s is acquired as the VA. Then the imaging processor reconstructs the image using VA.

Then in step S902, the sound speed determiner and the imaging processor set the values VA±10 m/s (1500 m/s and 1520 m/s) as the sound speed in a sub-region (sub-region B) adjacent to sub-region A, and generate the respective images.

In step S903, in the common region of sub-region A and sub-region B, the image of sub-region A with sound speed 1510 m/s is compared with the image of sub-region B with sound speed 1500 m/s and the image of sub-region B at sound speed 1520 m/s respectively. As the image characteristic amount used for comparison, the shift amount between the images is used. In this case, it is preferable to use the shift amount of the characteristic image of the absorber in order to perform an accurate comparison. It is assumed that a known image identification algorithm can be used for the comparison, and the shift amount is determined as an amount with which normalized cross-correlation becomes high.

Here the edge sharpness of the image of sub-region B is assumed to be the image characteristic amount P_{B}. The shift amount, which is the image characteristic amount of the common region of sub-region A and sub-region B, is assumed to be P_{AB}. The image characteristic amount P, which combines the image characteristic amounts P_{B} and P_{AB}, is defined as the following Expression (2) (step S904).

P=P_{B}−αP_{AB} (2)

Here α denotes a weight coefficient. In this example, it is assumed that α=0.3.

As the initial sound speeds of sub-region B are 1500 m/s and 1520 m/s, the sound speed with which P becomes largest in this range is assumed to be the optimum sound speed VB in sub-region B, using a gold section method (step S905).

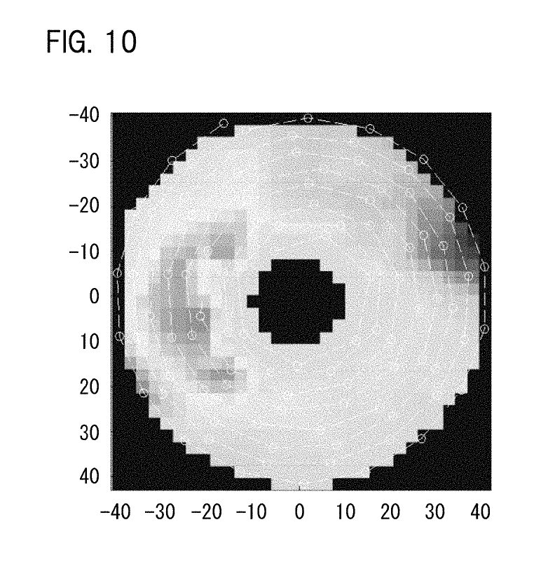

In step S907, it is determined whether the sound speed was determined for all sub-regions. For example, if the sound speed in sub-region C, which is adjacent to sub-region B, is not yet determined, this optimum sound speed VC is also determined using the same method. In other words, a gold section method using the same image characteristic amount expression, as mentioned above, is used in the optimum sound speed VB±10 m/s range in sub-region B. Thus in such sub-regions which are adjacent to each other, the optimum sound speed is determined repeatedly. FIG. 10 shows the calculated sound speeds.

As described above, in this example, both the processing to determine the optimum sound speed from the image characteristic amount in each sub-region, and a processing to determine the optimum sound speed in an adjacent sub-region based on the image characteristic amount in the common portion can be executed in a series of the flow. As a result, the number of times of the imaging process can be reduced to about one fourth that in Example 1. Therefore such effects as a reduction in processing time, a decrease of burden on the testee and user thereby, and cost reduction with respect to the operation capability, can be acquired. Further, the effect of the present invention, that is, acquiring the optimum sound speed in each segment of the object, can be maintained, and a high resolution image of the inside of the object can be acquired.

Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-226950, filed on Nov. 7, 2014, which is hereby incorporated by reference herein in its entirety.

1. An object information acquiring apparatus, comprising:
a region setter configured to set a plurality of sub-regions including at least a first region and a second region, which has a common region with the first region, in a region including a region of interest inside an object;
a sound speed determiner configured to set a plurality of sound speeds for the first region and for the second region respectively;
an imaging processor configured to
acquire an image of the first region using an electrical signal corresponding to an acoustic wave propagated from the object that is irradiated with light and received by a plurality of probes, with respect to each of the plurality of sound speeds set for the first region, and
acquire an image of the second region using the electrical signal with respect to each of the plurality of sound speeds set for the second region;
a characteristic amount acquirer configured to acquire a comparison characteristic amount, which is a characteristic amount in the common region of the image of the first region and the image of the second region, for each of the plurality of sound speeds; and
an information acquirer, wherein
the sound speed determiner acquires first sound speeds of the first region and the second region respectively using the comparison characteristic amount in the common region, and
the information acquirer acquires specific information on the object based on the electrical signal and the first sound speeds in the first region and the second region.

2. The object information acquiring apparatus according to claim 1, wherein the sound speed determiner acquires an individual characteristic amount from each image in the first region, acquires a first sound speed in the first region using the individual characteristic amount, acquires an individual characteristic amount from each image in the second region, and acquires a first sound speed in the second region by comparing the individual characteristic amount based on the first sound speed in the first region and the individual characteristic amount in the second region.

3. The object information acquiring apparatus according to claim 1, wherein the characteristic amount acquirer acquires, as the comparison characteristic amount, at least any of a shift amount of the images, a difference of pixel intensity, and a correlation index value between the first region and the second region.

4. The object information acquiring apparatus according to claim 3, wherein the characteristic amount acquirer uses a normalized cross-correlation value as the correlation index value.

5. The object information acquiring apparatus according to claim 1, wherein the sound speed determiner sets the plurality of sound speeds by changing only a predetermined value from the first sound speeds acquired for the first region and the second region respectively.

6. The object information acquiring apparatus according to claim 1, wherein the information acquirer creates an image representing the specific information on the common region by combining the specific information on the first region and of the second region in the common region.

7. The object information acquiring apparatus according to claim 1, wherein when acquiring the images of the first region and the second region, the imaging processor uses the electrical signals acquired under different conditions respectively.

8. The object information acquiring apparatus according to claim 7, wherein when acquiring the images of the first region and the second region, the imaging processor uses the electrical signals acquired using different probe patterns respectively.

9. The object information acquiring apparatus according to claim 7, wherein when acquiring the images of the first region and the second region, the imaging processor uses the electrical signals acquired by different light irradiations respectively.

10. The object information acquiring apparatus according claim 1, wherein when acquiring the images of the first region and the second region, the imaging processor weighs the electrical signal, which is acquired by each of the probes, differently.

11. The object information acquiring apparatus according claim 1, further comprising a scan drive unit configured to move the plurality of probes with respect to the object, wherein the information acquirer acquires the specific information based on the electrical signal, which the plurality of probes have acquired at each position to which the scan drive unit moved each of the probes respectively.

12. The object information acquiring apparatus according claim 1, wherein the information acquirer calculates the specific information on a portion, which the region setter has not set as the sub-region, out of the region of interest, based on the acquired specific information on a sub-region adjacent to this portion.

13. The object information acquiring apparatus according claim 1, further comprising a display unit displaying an image of the interior of the object based on specific information acquired by the information acquirer,
wherein the display unit display a boundary of the sub-regions.

14. The object information acquiring apparatus according to claim 13, wherein the display unit displays at least one of the first sound speed and the second sound speed for each of the sub-regions.

15. The object information acquiring apparatus according to claim 1, further comprising the light source.

16. The object information acquiring apparatus according to claim 1, further comprising the plurality of probes, wherein the plurality of probes is configured to receive at least each of an acoustic wave originating from the first region and an acoustic wave originating from the second region.

17. An information processing method comprising:
a region setting step of setting a plurality of sub-regions including at least a first region and a second region, which has a common region with the first region, in a region including a region of interest inside an object;
a sound speed determining step of setting a plurality of sound speeds for the first region and for the second region respectively;
a first acquiring step of acquiring an image of the first region using an electrical signal corresponding to an acoustic wave propagated from the object that is irradiated with light and received by a plurality of probes, with respect to each of the plurality of sound speeds set for the first region,
a second acquiring step of acquiring an image of the second region using the electrical signal with respect to each of the plurality of sound speeds set for the second region;
a characteristic amount acquiring step of acquiring a comparison characteristic amount, which is a characteristic amount in the common region of the image of the first region and the image of the second region, for each of the plurality of sound speeds; and
a information acquiring step,
wherein the sound speed determining step includes acquiring first sound speeds of the first region and the second region respectively using the comparison characteristic amount in the common region, and
wherein the information acquiring step includes acquiring specific information on the object based on the electrical signal and the first sound speeds in the first region and the second region.

18. A non-transitory computer-readable storage in which a program is stored, the program being for causing a computer to perform the information processing method according to claim 17.
